(19) United States
(12) Patent Application Publication
(10) Pub. No.: US 2020/0171509 A1
(43) Pub. Date: Jun. 4, 2020

(54) DEVICES AND METHODS FOR ONE-STEP STATIC OR CONTINUOUS MAGNETIC SEPARATION

(71) Applicant: BIOMAGNETIC SOLUTIONS LLC, State College, PA (US)

(72) Inventor: Paul A. Liberti, State College, PA (US)

(21) Appl. No.: 16/614,501

(22) PCT Filed: Apr. 2, 2018
(86) PCT No.: PCT/US2018/025669

§ 371 Date: Nov. 18, 2019

Related U.S. Application Data
(60) Provisional application No. 62/480,397, filed on Apr. 1, 2017.
(60) Provisional application No. 62/529,574, filed on Jul. 7, 2017.
(60) Provisional application No. 62/546,700, filed on Aug. 17, 2017.
(60) Provisional application No. 62/591,833, filed on Nov. 29, 2017.

(51) Int. Cl. B03C 1/01; G01N 33/543

(52) U.S. Cl. CPC B03C 1/01; B03C 2201/26; B03C 2201/18; G01N 33/54333

(57) ABSTRACT

This invention relates to the static or continuous magnetic separation of specific entities from mixtures where the separation of target entities is clone in a one-step process. It is universally applicable to the harvest or removal of such entities for processing of biological molecules, cells of all types, virus particles, and the like, and for small- to large-scale separations of the same. By manipulation of the scientific principles that underlie this invention, specific targets can be conveniently captured for analysis, harvested or subjected to further processing on a collection surface free of bystander components. The principles employed and the methods disclosed completely obviate the need for washing of targeted entities such as cycles of resuspension and magnetic separation for removal of contaminating substances in the case of static separation or complex cycles of sample introduction and harvest to perform continuous processing.

### CROSS-REFERENCE TO RELATED APPLICATIONS

This application claims priority under 35 U.S.C. § 119(e) to U.S. Provisional Patent Application Nos. 62/480,397 filed on Apr. 1, 2017; 62/529,574 filed on Jul. 7, 2017; 62/546,700 filed on Aug. 17, 2017; and 62/591,833 filed on Nov. 29, 2017 which are hereby incorporated by reference in their entirety.

### FIELD OF THE INVENTION

This invention relates to the static or continuous magnetic separation of specific entities from mixtures where the separation of target entities is done in a one-step process. It is universally applicable to the harvest or removal of such entities for bioprocessing of biological molecules, cells of all types, virus particles, and the like, and for small- to large-scale separations of the same. By manipulation of the scientific principles that underlie this invention, specific targets can be conveniently captured for analysis, harvested or subjected to further processing on a collection surface free of bystander components. The invention also employs newly discovered properties of a class of magnetic nanoparticles that enable these materials to be used to perform one-step static separations and enable continuous magnetic separations. The principles employed and the methods disclosed completely obviate the need for washing of targeted entities such as cycles of resuspension and magnetic separation for removal of contaminating substances in the case of static separation or complex cycles of sample introduction and harvest to perform continuous processing.

### BACKGROUND OF THE INVENTION

Magnetic separations in industrial applications and in biological systems are well known in the art. In the case of removing ferromagnetic contaminants from dry mixtures or slurries in a variety of manufacturing processes, simple solutions for continuous operations are well known. On the other hand, in biological systems, truly continuous devices and protocols for magnetic separations that yield high purity product at reasonable yields have not, in fact, been realized.

To create a continuous separation process in its simplest form for the above-mentioned biological entities, leveraging an intrinsic property of the entities to be separated has been the most successful route. For example, owing to their different sizes but relatively similar-sized nuclei, mammalian cells have differing densities, with the smaller cells being denser. Thus cells of different sizes are readily separated in density gradients by centrifugation. The continuous introduction of cell mixtures into specialized centrifuge heads containing density gradients and the continuous removal of a density layer containing desired cells is well known in the art. Also well known in the art is free-flow electrophoresis for the separation of proteins and macromolecules. In that case, a stream containing a mixture of proteins or macromolecules is introduced at some position near the top of a suitable vessel containing appropriate flowing electrolyte and with an electric potential placed on its sides. If the sample contains materials of differing electrophoretic mobility, the different species will move apart from each other as they flow downwards and can be harvested from different ports along the bottom of the apparatus. Each of the foregoing examples is enabled by the fact that one or more entities of interest can be moved out of a mixture or away from the other species therein effectively by a single step.

Positive selection magnetic separation for cells and other biological entities are typically done by batch methods. For separations done in vessels (e.g., tests tubes, beakers, bags), several process steps must be performed to obtain pure product. In magnetic separations, such steps are typically done in 2-3 cycles to obtain purified product, and could comprise the following steps: 1) magnetically labeled entities are pulled to the side of the vessel; 2) supernatant containing unlabeled entities is removed and discarded; 3) the vessel is removed from the magnetic gradient; 4) wash buffer is added; 5) entities are re-suspended; and 6) the re-suspended entities are again magnetically separated. In the case where magnetic labeling is such that high-magnetic-field-gradient columns are employed, the process could be as follows: 1) the mixture containing magnetically labeled entities is passed through an appropriate column in a magnetic field; 2) labeled entities magnetically adhere to the column; 3) the column is washed free of sample and unlabeled entities that might have been trapped in the column; 4) the column is removed from the magnetic field; and 5) cells are recovered by passing buffer through the column, sometimes with the augmentation of vibration of the column. For both of these systems, not only are they complicated by their many steps, they do not lend themselves to continuous separation.

Many inventions address attempts to create continuous magnetic separation systems or, in some cases, one-step magnetic separation procedures by employing a variety of means toward those ends. Those works are summarized below:

Ching-Jen, et al.'s patents (U.S. Pat. Nos. 6,129,848A, 6,132,607A and U.S. Pat. No. 6,036,857A) describe methods for the continuous separation of chemicals, cells or components from blood (e.g., WBCs). Ching-Jen, et al.'s methods represent a series of batch-mode separations to effect a continuous separation (i.e., discontinuous or batch processing).

U.S. Pat. No. 4,910,148 to Sorenson, et al. relates to a method and device for separating magnetized particles from biological fluids, particularly white blood cells using a monoclonal antibody to link the cells to magnetic beads. Sorenson's separation is static (i.e., no flow) and is conducted in a plastic blood bag. The magnetic beads are linked to malignant white blood cells by an agitation process and then a magnetic field is applied to keep the white blood cells bearing magnetic beads in the disposable plastic bag. The Sorenson device also requires space between the magnets, which does not optimize the magnetic force. The back plate of the Sorenson device is a soft magnetized material and the magnets are samarium-cobalt. Sorenson has a volume limitation since it uses a blood bag (150 mL) and there is no decoupling between the beads and the white blood cells. Further, the cells remain in the disposable blood bag after separation.

U.S. Pat. No. 5,514,340 to Lansdorp, et al. relates to a device for separating magnetically labeled cells in a sample using an applied magnetic field. Lansdorp uses magnetized screens to attract the magnetic particles allowing the biological fluid to be caught in the magnetic wires of the screen. The magnets used in Lansdorp must constantly be cleaned since there is contact between the magnets and the blood cells.

U.S. Pat. No. 5,567,326 to Ekenberg, et al. relates to an apparatus and methods for separating magnetically responsive particles from a non-magnetic test medium in which the magnetically responsive particles are suspended. In Ekenberg, small patch amounts of biological fluid are placed in a tube then a magnetic pin is inserted in the fluid for separation.

U.S. Pat. No. 4,988,618 to Li, et al. relates to a magnetic separation device for use in immunoassay or hybridization assay procedures. The Li device comprises a base having a plurality of orifices for receiving non-ferrous containers which hold the sample and the assay components, including ferrous particles. The orifices are surrounded by a plurality of magnets which are spaced about the peripheral of the orifices.

U.S. Pat. No. 4,935,147 to Ullman, et al. relates to a method for separating a substance from a liquid medium, particularly applicable for separation of cells and microorganisms from aqueous suspension, but also for the determination of an analyte. Although Ullman discusses a method with a reversible non-specific coupling, the method is not continuous nor does it utilize a multi-dimensional gradient.

U.S. Pat. No. 5,968,820 to Zborowski et al describes a quadrupole based continuous separator that employs laminar flow of magnetically labeled sample adjacent to flowing buffer to effect continuous separation. No performance data is given. However, the system has been employed for the isolation of clustered pancreatic islet cells by Weegman et al [J. Diabetes Res. 2016, Article ID 6162970, (2016)]. That system can only give high purity at very low concentrations which is a significant limitation.

U.S. Pat. No. 5,541,072 to Wang et al. describes a continuous feed separator that captures target cells in a hydrodynamically designed flow cell placed between arrays of alternating bucking magnets. That system proved very effective for negative selection, whereas positively selected cells were difficult to recover. In the work leading up to that disclosure, attempts were made to employ a two vector system—unidirectional downwards flow and a strong magnetic gradient, similar in concept to '820—to create a continuous separation system. The notion employed was analogous to free flow electrophoresis where a stream of mixed proteins is directed into a downward flowing rectangular column while a strong electric potential is exerted in the horizontal direction. In that system, proteins of high charge/mass ratio rapidly separate from the stream and can be harvested because of their lateral displacement from the original stream.

When that simple notion was explored for a ferrofluid-based system (a system employing highly magnetic colloidal nanoparticles such as those described by Liberti et al. in U.S. Pat. Nos. 5,597,531 and 5,698,271), it was found that when a stream of cells labeled with magnetic nanoparticles (90-140 nm) was introduced into a rectangular volume of downwardly flowing buffer, instead of magnetically labeled cells being pulled out of the stream towards the higher gradient region and becoming separated from non-target cells, the entire stream moved as a phase towards the magnetic gradient that was applied to the system. To further understand this behavior, '072 discloses the results of an in-depth series of experiments to explore our discovery of this phenomenon, which was referred to as Ferro-phasing. Just like the streaming experiments previously mentioned, when a droplet of magnetic nanoparticles mixed with food coloring was introduced into a magnetically inert fluid such as water in a microtiter well which was positioned in a magnetic quadrupole device, the colored liquid and the magnetic nanoparticles (i.e., ferrofluid) immediately formed an annular cylinder distributed around the periphery of the microtiter well while the water formed a clear cylinder within the annulus. In other words, the ferrofluid/food dye mixture moved as a unitary phase to the regions of highest magnetic field gradient. Over time, the ferrofluid separated to the wall of the vessel, leaving behind a diffuse ring of food coloring. Importantly, the initial phenomenon clearly demonstrates that the food coloring acted like it was incorporated into a phase. If instead the food coloring was first mixed with the water and ferrofluid within the microtiter well and subsequently placed in a quadrupole separator, ferrofluid separated to the wall, leaving the food coloring behind.

One hypothesis offered to explain those experiments could be a consequence of the fact that ferrofluids and other magnetic nanoparticles are known to form long chains under the influence of magnetic field gradients (Liberti, unpublished observations, Ugelstad also). It can be shown that 8 μg (based on Fe mass) of 130 nm particles that are about 80% magnetite placed in a 1 cm³ chamber could form about 30,000 linear chains. Presuming that such strands would likely align parallel to each other, one could imagine that because of their highly hydrophilic nature, they would strongly interact with neighboring water molecules, resulting in a gel-like structure. Evidence of this gel-like structure, or Ferro-phase as it is referred to, is provided in U.S. provisional application No. 62/546,700 where it is disclosed how this phenomenon can be used to move or position non-magnetic entities such as small molecules, macromolecules, and cells contained therein.

Based on the observations that a two-phase system, comprising an inert fluid and a Ferro-phase, can be formed and maintained, it was concluded in '072 that a simple approach to magnetic separation analogous to free-flow electrophoresis using colloidal nanoparticles is not feasible. As noted, '072 does disclose an invention for large-scale continuous separation, but the need to further process collected target cells because of entrained non-target cells limits the invention.

The discoveries made and disclosed herein, as well as those previously disclosed in U.S. provisional application 62/480,397, tend to show that the conclusion made in '072 as regards the use of colloidal magnetic nanoparticles for doing one-step static or continuous magnetic separations was incorrect. Our recent discoveries on how Ferro-phasing can be overcome by density adjustments make such separations possible.

### SUMMARY OF THE INVENTION

The present invention and that of provisional application U.S. 62/480,397 overcome the aforementioned problems, regarding the inability to create a magnetic separation system analogous to free-flow electrophoresis employing colloidally stable magnetic nanoparticles, by an effective and simple means for overcoming counteracting Ferro-phasing. It was discovered that Ferro-phasing can be counteracted by adjusting the density of the ferrofluid-containing phase, the non-ferrofluid-containing phase, or both. For example, if in a microtiter well, a ferrofluid-containing solution (about 3-10 μg Fe/mL) is layered over a buffer containing 1% sucrose, those layers will be stable over long periods of time. For the purposes of this invention, the term “layer” refers to a layer of medium or the like. On the other hand, if the microtiter well is placed over a downward-pulling magnetic device, the upper ferrofluid layer (more easily visualized by the inclusion of small amounts of food dye) will immediately move downward as a phase towards the magnet and become layered under the sucrose-containing buffer. If the well is subsequently moved off the magnet, the phases will revert to their original positions. It is notable that these phenomena can be repeated several times. On the other hand, if the sucrose level of the lower buffer layer is increased to 5% and the well is placed on the magnet, the phases will not move, though the ferrofluid will begin to move through the sucrose-containing lower layer toward the magnet. Thus Ferro-phasing can be overcome by adjusting the density of the solutions in accordance with the direction of the magnetic gradient. If target cells are being pulled upwards through a non-magnetic phase, the density of the lower ferrofluid-containing phase needs to be increased, with the degree of increase being related to the ferrofluid concentration. Thus a bottom layer of ferrofluid-containing solution with 0.5% sucrose overlaid with buffer when placed under an upward-pulling magnetic device will Ferro-phase such that the lower layer will move as a phase and replace the top layer.

By making such density adjustments, it has been discovered that it is possible to magnetically separate target cells out of a ferrofluid-containing phase, such that the target cells leave the phase that originally contained the ferrofluid solution and enter the non-ferrofluid-containing phase. Most importantly, it has been discovered that the non-ferrofluid-containing phase serves to effectively “wash” target cells free of non-target cells, giving exceptional purities in one step. This “washing” effect is confirmed by experiments which show that the height of the column of the non-ferrofluid-containing phase through which target cells traverse is proportional to the purity of the product.

### DESCRIPTION OF THE DRAWINGS

FIG. 1 shows a vessel 1 containing a buffer layer 2 overlaid atop a denser cell-containing solution 3. A removable collection plate 4 is placed on top of the vessel 1 to capture cells which come into contact with it, and a magnetic device 5 is placed on top of the collection plate 4 to pull magnetically labeled cells toward the collection plate 4.

FIG. 2 depicts a manual system wherein separations can be performed by concepts illustrated in FIG. 1. A vessel 1 containing a buffer layer 2 overlaid atop a denser cell-containing solution 3 is placed onto a base 6. A magnetic device 5 is mounted on a platform 7, which can be raised or lowered on guiding posts 8 of the base 6. The underside of the platform 7 has grooves 9 which can accept an appropriately flanged receiving chamber 10 to mount the receiving chamber 10 near the magnetic device 5.

FIG. 3 illustrates a device used for performing continuous magnetic separations. A dense cell-containing solution 3 is introduced into an elongated chamber 11 through the bottom left inlet port 12 and pumped out through the bottom right outlet port 13. A less dense wash buffer 2 is introduced through the top left inlet port 14 and pumped out through the top right outlet port 15. Due to the differing densities, the two solutions do not mix within the chamber, forming a distinct boundary 16 between the wash buffer 2 and the cell-containing solution 3. A magnetic device 5 is used to pull magnetically labeled cells out of the denser phase 3 and into the less dense phase 2 such that they are pumped out of the chamber 11 with the wash buffer 2.

FIG. 4 depicts a similar device to that shown in FIG. 3, wherein the elongated chamber 17 has a removable collection plate 4 capable of binding cells upon contact. In this device, magnetically labeled cells are pulled to the collection plate 4 before they can exit the chamber 17.

FIG. 5 shows the cross section of a similar device to that shown in FIG. 4, wherein the chamber 18 has tapered sides 19, 20 that force magnetically labeled entities to be directed toward the center as they move towards the removable collection plate 4.

### DETAILED DESCRIPTION OF THE INVENTION

Static One-Step Magnetic Separations

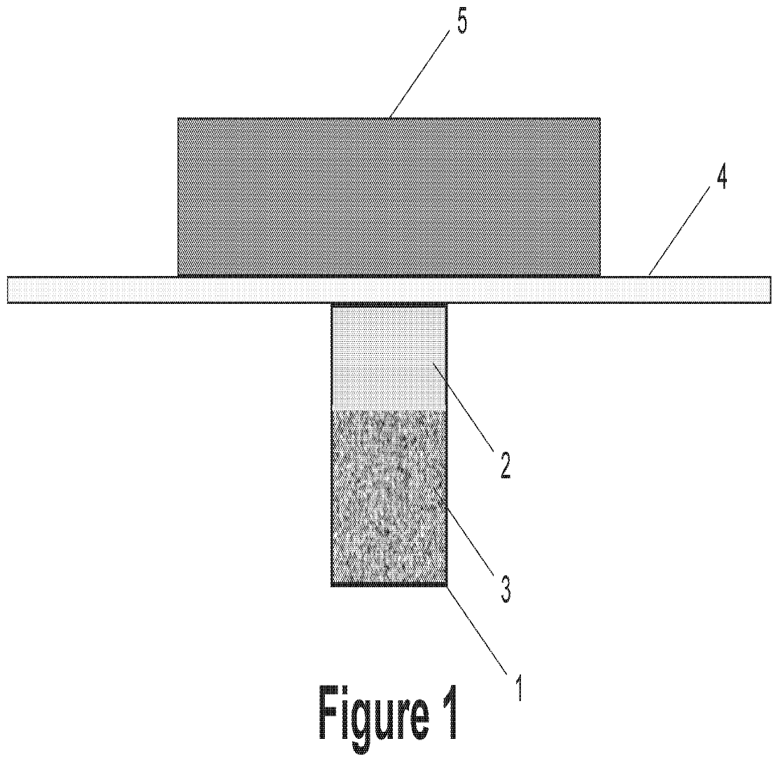

To evaluate the parameters of the herein disclosed two-phase separation system (i.e., a magnetic phase and a non-magnetic phase), extensive experiments were performed in microtiter wells wherein a solution containing ferrofluid-labeled cells was used as the bottom layer. A volume of buffer solution was layered on top of the bottom layer such that the well volume was slightly exceeded, forming a convex meniscus; in this way, when a slide was placed on top of the well, a small amount of buffer spilled out of the well, but no air gap was present between the buffer solution in the well and the slide. This arrangement is illustrated in FIG. 1, wherein a vessel 1 contains a buffer layer 2 overlaid atop a denser cell-containing solution 3. A removable collection plate 4 is placed on top of the vessel 1 to capture cells which come into contact with it, and a magnetic device 5 is placed on top of the collection plate 4 to pull magnetically labeled cells toward the collection plate 4. For many of these experiments, a polylysine-coated slide was used as the collection plate 4 so that target cells brought to the undersurface of such slides by an upward-pulling magnetic device would adhere, thereby facilitating quantitation. For such experiments using microtiter wells, the maximum height of the column of liquid (i.e., buffer layer 2 and cell-containing solution 3) was 11 mm. Initially, atop 9-10 mm columns of cell-containing solution 3, buffer layers 2 were added to fill the vessel to the top, polylysine-coated slides were placed atop the filled vessels, and a bucking magnet device constructed by abutting like poles (i.e., N—N or S—S) of rare-earth magnets against a 7 mm soft iron spacer—an arrangement capable of producing high magnetic field gradients—was placed above the slide such that the iron spacer (i.e., the region of highest magnetic field gradient) nearly spanned the vessel opening. Such arrangements create gradients that quite uniformly collect magnetic entities over the 7 mm width of the soft iron spacer. As disclosed in US Patent Provisional Ser. No. 62/489,397, after a 10 min exposure to the upward-pulling magnetic gradient, 90% of target cells were determined to be affixed to the slide. Furthermore, there was no evidence of non-target cells—including human red blood cells, when they were included in the cell mixture—on the slide. Thus magnetically labeled cells passing through the buffer layer are “washed” and non-target cells do not follow them through the buffer layer. For this simple system, not only does it demonstrate that a one-step immuno-magnetic separation is possible, but it also presents the opportunity to conveniently perform multiple operations on the target cells affixed to the polylysine-coated slides—a considerable advantage.

To determine the extent of “washing” by the buffer layer that is required to obtain high purities, further experiments were performed in microtiter wells wherein the height of the buffer column was varied (total column height was fixed at 11 mm). HPB cells (human-derived T cell line; CD3+) were labeled with biotinylated anti-CD3, subsequently labeled with streptavidin-functionalized ferrofluid, and spiked into buffer containing 5% sucrose. Cell mixtures were placed into microtiter wells with column heights of 2, 3, 4 and 5 mm, and appropriate volumes of sucrose-free wash buffer were subsequently layered on top of each cell mixture to fill each microtiter well to its maximum capacity. Polylysine-coated slides were placed on top of each filled vessel, and a bucking magnet device was used to provide a magnetic field gradient for 10 min. In each case, it was determined that target cells could be pulled through the various heights of wash buffer and that they were recovered essentially quantitatively (estimated by counting cells adhered to the slides). We then tested mixtures of magnetically labeled HPB cells (pre-stained with a red intracellular stain for identification) and unlabeled U937 cells (human-derived CD3− monocyte cell line; pre-stained with a green intracellular stain for contrast). It was determined that only the magnetically labeled HPB cells were pulled through the buffer layer and that no U937 cells were captured. Following these experiments, human red blood cells were spiked into the system at levels as high as 15% hematocrit, and again, only HPB cells were captured on the polylysine-coated slides.

Based on the ability to pull magnetically labeled target cells up from the ferrofluid layer, through the buffer layer in a highly purified state, and immobilize them on a polylysine-coated surface, attempts were made to recover cells from the collection surface. This was accomplished by placing cell mixtures of appropriate density containing ferrofluid-labeled cells into microtiter wells and layering less dense buffer on top. A Parafilm-wrapped, upward-pulling magnetic device was then placed atop the well in direct contact with the buffer layer. After 10 min of magnetic separation, the Parafilm-wrapped to magnetic device was lifted off the well, and the droplet adhering to the Parafilm was recovered and examined microscopically. With the demonstration that target cells could be recovered in a highly purified state, a more sophisticated system was created that would allow qualitative and quantitative analysis of product.

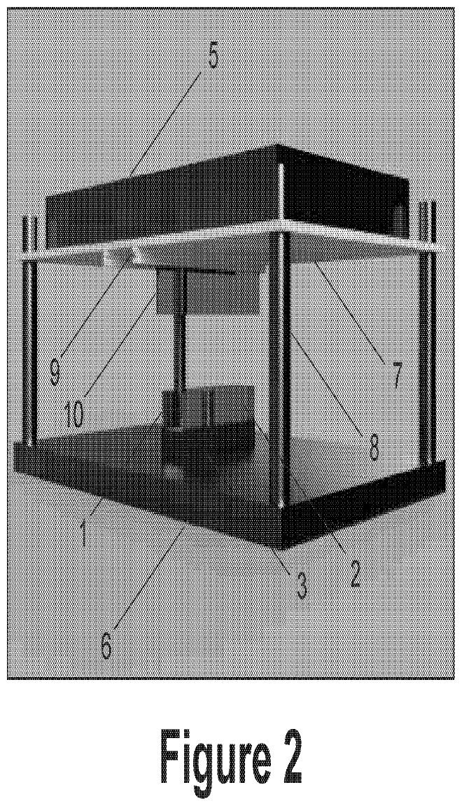

FIG. 2 depicts a manual system that was used to demonstrate the one-step nature of the separations disclosed herein. A vessel 1 containing a buffer layer 2 overlaid atop a denser cell-containing solution 3 is placed onto a base 6. The vessel 1 might have internal dimensions of 1 cm wide by 4-5 cm long by 1.5-3 cm deep. A magnetic device 5 which exerts an upward-directed magnetic field gradient is mounted on a platform 7, which can be raised or lowered on guiding posts 8 of the base 6. The underside of the platform 7 has grooves 9 which can accept an appropriately flanged receiving chamber 10 to mount the receiving chamber 10 near the magnetic device 5 and allow them to be moved in tandem. The internal dimensions of the receiving chamber 10 allow it to be placed loosely over the vessel 1 such that any excess liquid from the buffer layer 2 that might overflow when the receiving chamber 10 is placed on top of the vessel 1 will not be trapped between the adjacent walls of the two chambers.

To perform a one-step magnetic separation using the device of FIG. 2, the vessel 1 is placed onto the base 6 with the magnetic device 5 removed. The vessel 1 is loaded with a cell-containing solution 3 comprising ferrofluid-labeled cells, with its density appropriately adjusted so as to overcome Ferro-phasing. The buffer layer 2 is then placed on top of the cell-containing solution 3 with an appropriate volume to form a convex meniscus. The receiving chamber 10 is loaded into the platform 7, and the platform 7 is lowered on the guiding posts 8 to bring the receiving chamber 10 into contact with the vessel 1. With the magnetic device 5 in close proximity to the vessel 1, magnetic separation is allowed to take place for an appropriate interval (typically 8-15 min). During that time period, target cells will have been pulled upwards out of the lower layer, through the buffer layer 2, and onto the underside of the receiving chamber 10 such that when the platform 7 is raised up and away from the base 6, the magnetically labeled target cells are retained on the underside of 10. The platform 7 is then removed from the base 6 and rotated 180° so that the appropriate solution (e.g., buffer) can be added to the receiving chamber 10. The receiving chamber 10 can be removed from the platform 7 and moved away from the magnetic device 5 to re-suspend the target cells. Alternatively, while the receiving chamber 10 is still engaged with the platform 7, it may be desirable to perform various reactions or other procedures on the magnetically immobilized cells.

To perform a separation with direct immuno-magnetic labeling, a cell-containing solution 3 is incubated with ferrofluid (in the vessel 1, if desired), to which appropriate targeting molecules are coupled (e.g., monoclonal antibodies or other recognition molecules). In the case where an indirect immuno-magnetic labeling method is used, appropriate targeting molecules are incubated with the cell-containing solution 3 for an appropriate interval (5-15 min) and unbound targeting molecules are removed by various methods well known in the art. In many cases when employing ferrofluids, this removal step is unnecessary and the ferrofluid can be directly added, initially mixed, and allowed to bind. Since ferrofluid binding to targets is not affected by continual mixing (diffusion-controlled reaction), samples can be placed directed into the vessel 1 and positioned on the base 6. While incubation is taking place, the layering process can be performed. As no wash or re-suspension steps are required by this invention, target cells can be separated in as little as 20 min when employing direct labeling. For the indirect method, an additional 10-15 min would be required.

It should be understood that a system similar to that which is depicted in FIG. 2 can be readily implemented in parallel to allow multiple samples to be processed simultaneously. To construct such a system, the base 6 would be modified to accept a plurality of vessels 1, and the platform 7 would be modified to comprise multiple sets of grooves 9 to accept a plurality of receiving chambers 10. The magnetic device 5 would also need to be modified to exert a magnetic field gradient at intervals along the device, corresponding to the locations of the receiving chambers 10. All of these modifications are straightforward, and we have constructed a working prototype of the appropriate magnetic device 5, which is simply an array of bucking magnets.

It should also be noted that the system depicted in FIG. 2, and particularly the foregoing system capable of processing multiple samples simultaneously, would benefit greatly from automation. Employing a multi-stage peristaltic pump to automate the layering process would be ideal, and automation of the platform movement through incorporation of linear and rotary actuators would likely improve reproducibility.

To determine if larger magnetic particles which do not exhibit Ferro-phasing could be used in the single-step separation process disclosed herein, we examined 2.8 μm streptavidin-coated Dynabeads® (Dynabeads® M280 Streptavidin, Thermo Fisher Scientific) in a static separation. Cells that had been labeled with biotinylated antibody, subsequently labeled with streptavidin-coated Dynabeads®, and densified with sucrose were placed into a vessel 1. This dense cell-containing solution 3 was overlaid with a buffer layer 2, a collection plate 4 was placed atop the vessel 1, and an upward-pulling magnetic device 5 was placed atop the collection plate 4 as described above for the ferrofluid-based system. We determined that a one-step separation can be achieved, yielding high purity target cells. For the device used, yields were about 20% less than with ferrofluid, but this could likely be improved.

Comparing these two classes of magnetic particles, ferrofluids have some significant advantages over Dynabeads®. For example, ferrofluids are colloidal and their reactions are diffusion controlled, which allows the magnetic nanoparticles to remain suspended indefinitely and eliminates the need for mixing. In contrast, optimal reactions with Dynabeads® require mixing, and labeled cells must be processed in a timely manner to prevent settling. Nonetheless, the ability to incorporate density layering, where one layer contains a densified magnetically labeled mixture in contact with a less dense “washing” buffer which permits one-step separations without the need for additional cycles of re-suspension and re-separation, has wide utility.

Continuous Magnetic Separation

Based on the ability to 1) magnetically pull cells out of a dense phase and upward through a less dense phase—or alternatively, magnetically pull cells out of a less dense phase and downward through a dense phase—and 2) pull target cells through a sufficiently large column of wash buffer, which is very effective at preventing non-target cells from reaching the collection surface, there is clearly the potential to use this finding to create a novel system for continuous magnetic separation providing the phases can be introduced into, flowed through, and collected from an appropriate vessel without significant mixing. Hence, by eliminating Ferro-phasing, two useful systems are created.

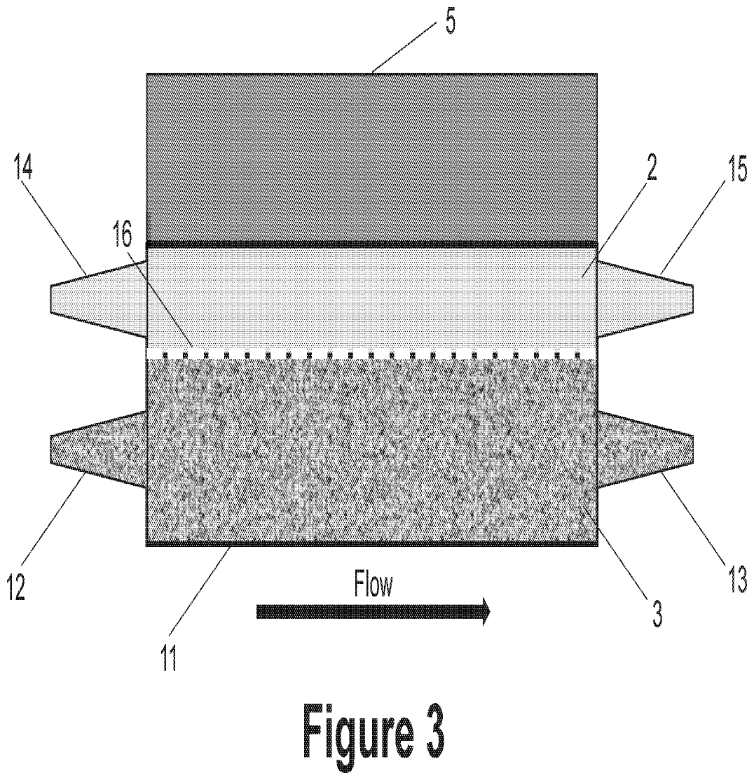

FIG. 3 depicts one system for performing continuous magnetic separation employing the concepts disclosed herein. Systems of this type are hereafter referred to as Trans-Density Magnetic Separators (TDMS). In this TDMS device, two solutions—wash buffer 2 and dense cell-containing solution 3—flow (in this case, from left to right) through an elongated chamber 11. The denser solution 3 containing a cell suspension with magnetically labeled target cells is introduced into the chamber 11 through the inlet port 12 and pumped out through the outlet port 13. Inlet port 14 allows the less dense wash buffer 2 to be introduced into the chamber 11, which exits the chamber 11 through outlet port 15. The boundary 16 between the wash buffer 2 and the cell-containing solution 3 is maintained by the differing densities and the laminar flow regime, which prevents mixing. A magnetic device 5 positioned above the chamber 11 imparts an upward-directed magnetic field gradient to pull magnetically labeled cells out of the denser phase 3 and into the less dense phase 2 such that they are pumped out of the chamber 11 with the wash buffer 2.

To perform a continuous separation, the TDMS device can be conveniently loaded with the two liquids of different densities such that a distinct boundary 16 between them is established and maintained. This can be accomplished by pumping the denser of the two liquids (not containing the cell mixture) at a controlled rate into the chamber via inlet port 12 to fill the chamber 11 to a defined level (indicated by the dashed line in FIG. 3 representing the boundary 16), after which the solution is pumped out through outlet port 13 at the exact same rate. Subsequently, the less dense wash buffer 2 is pumped into the chamber through inlet port 14 until it exits through port 15; we have found that the denser liquid can be stationary or flowing during this process. Once flow equilibrium is achieved with both liquids moving through the chamber at the same rate and with no disturbance at the interface, the dense cell-containing solution 3 can be introduced.

As a cell-containing solution 3 flows to the right, magnetically labeled cells will move upwards in the chamber 11 tracing out an arc, the curvature of which will be a function of the speed with which the cells traverse the chamber 11, the densities and viscosities of the cell-containing solution 3 and wash buffer 2, the gradient of the magnetic field strength produced by the magnetic device 5, and each cell's magnetic load. It should also be clear that while the length of the chamber 11 will not affect the speed with which cells move upwards, the longer the chamber 11, the more rapidly the solutions can be flowed through it. By appropriately controlling flow, solution densities and viscosities, magnetic gradient, degree of magnetic labeling, and length of the chamber 6, targeted cells will move into the upper wash buffer layer 2 and exit through outlet port 15, where they can be harvested. It should be understood that there is essentially no limitation on the volume of cell suspension that can be processed through the TDMS device.

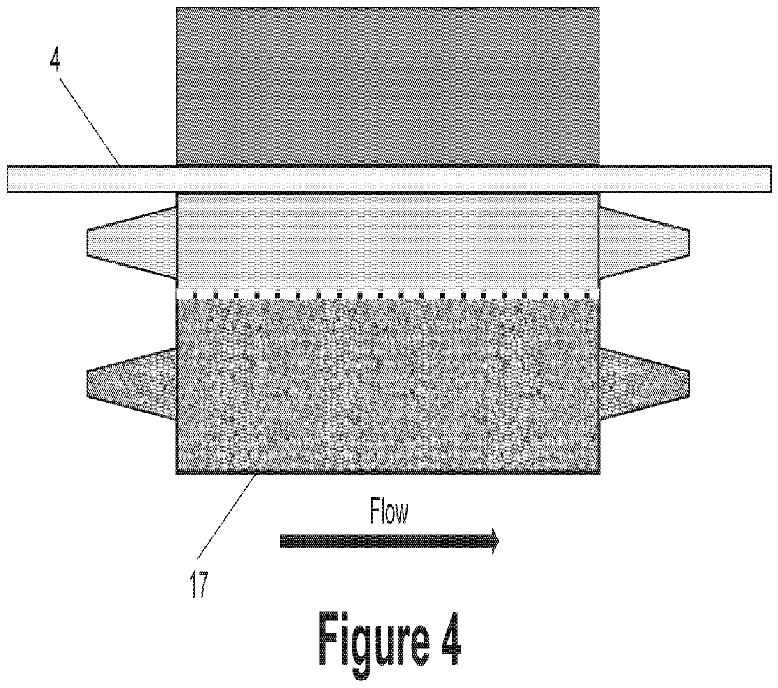

FIG. 4 depicts a similar TDMS device to that shown in FIG. 3, wherein the elongated chamber 17 has a removable collection plate 4. This collection plate 4 has an undersurface that binds cells upon contact, either by simply taking advantage of the high net negative charge on cells (i.e., non-specifically) or through some binding pair interaction (i.e., specifically). For the former, there are many surface coatings, such as polylysine or other polycations, aminosilane derivatives, and other positively charged moieties capable of strongly interacting with negatively charged cells. Specific binding pairs include cell surfaces receptors and specific antibodies or other binding proteins. Alternatively, the surface can be modified such that it interacts with an agent on the magnetic nanoparticle, forming a binding pair.

The primary purpose of the TDMS device depicted in FIG. 4 is the capture of magnetically labeled entities that are brought into proximity of the undersurface of the collection plate 4. The main difference between this TDMS device and the TDMS device depicted in FIG. 3 is that magnetically labeled entities have to be pulled to the top of the chamber 17 such that they have the opportunity to bind to the collection plate 4 before exiting the chamber 17. As compared to the system that is used to simply transfer magnetically labeled entities from one phase into another phase, one or more changes to the system are required, which could include: 1) increasing the dwell time of such an entity within the magnetic field, either through lowering the solution flow rate(s) or increasing the length of the chamber 17; 2) increasing the magnetic field gradient; or 3) increasing the degree of magnetic labeling. For the TDMS device of FIG. 4, the initial loading of the two phases would be similar to that described for the TDMS device of FIG. 3, and once the liquids of different densities are flowing appropriately, the sample would be introduced.

Since the arc that a magnetic entity makes as it moves toward the collection plate 4 where it binds is a function of the flow rate, solution density and viscosity, gradient of the magnetic field strength, and magnetic load of an entity, there are several manipulations that could be applied. For example, assuming a system of labeled entities which all have similar magnetic loads and are similar in size and shape, if the collection plate 4 is sufficiently long, those entities would be collected in a band along the length of the collection plate 4. If the direction of flow is from the left to the right, then increasing the flow rate should move the collected band to the right. Therefore if one desires to collect such entities along some particular length of the collection plate 4, the flow rate of the solutions would be increased (or decreased) as the separation proceeds. Thus the user can control the location and spread of magnetically captured entities.

On the other hand, should the magnetically labeled entities be heterogeneous as regards magnetic labeling, density, size, or shape, this will manifest in how they are distributed on the collection plate 4. Assuming a population of cells has a distribution of receptors, then regardless of the method of magnetic labeling (i.e., indirect or direct), their magnetic load would have an analogous distribution to the receptor distribution. As such, with appropriate control of the flow, solution properties, and magnetic field gradient, the collection pattern of such a population on the collection plate 4 would reflect its receptor distribution. That is, high-density-receptor cells with high magnetic loading would collect closer to the inlet than low-density-receptor cells with less magnetic loading. Hence, the TDMS device so described has analytical capabilities that are a direct result of the physics that the system imposes on magnetically labeled entities.

For a system where magnetic entities are to be captured on the collection plate 4, it may be desirable to confine them to a narrower band (i.e., in the dimension orthogonal to the direction of flow) as opposed to being spread across the entire width of the collection plate 4. To date, TDMS devices we have tested have had rectangular cross sections. Hence, with magnetic entities being pulled from one phase to the other phase, collected entities will be spread over the entire width of the collection plate 4. There are at least two means for narrowing the width of the collection band. One is to use a magnetic device whose gradient across the width of the collection plate 4 is non-uniform such that magnetic entities can effectively be collected in a narrow band. This can be achieved to simply by employing a bucking magnet arrangement where opposing magnets abut with no spacer (unlike the arrangement described above). In that case, the gradient of the magnetic field strength at the contact plane formed by the opposing magnets is extraordinarily high and non-uniform; in fact, this type of magnetic arrangement is well known to collect such magnetically labeled entities in a relatively narrow band. Alternatively, the cross section of the chamber 17 can be designed so as to force magnetically labeled entities to form a narrow band on the collection plate 4. It should be clear that both of these strategies can be employed in tandem to narrow the width of the collection band.

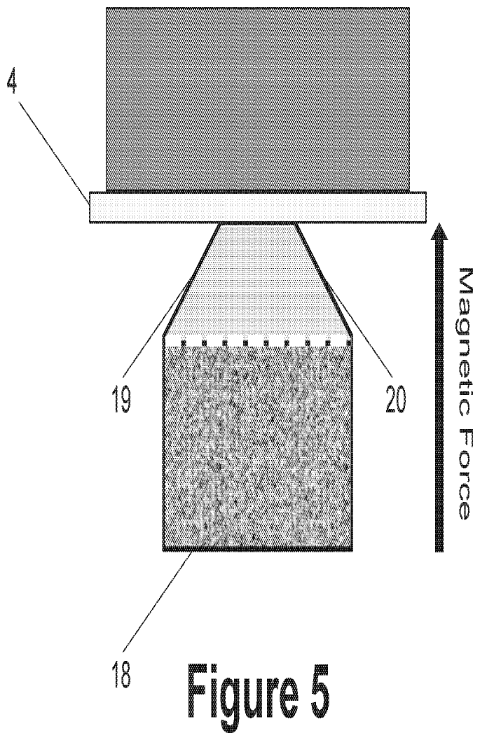

FIG. 5 shows the cross section of a TDMS device, wherein magnetically labeled entities are pulled upwards toward a removable collection plate 4 in a chamber 18 that has tapered sides 19, 20. The tapered sides 19, 20 of the chamber 18—in concert with the upwardly pulling magnetic force—will cause magnetically labeled entities that are off-center to be directed towards the center of the chamber 18 as they move towards the collection plate 4. Unlike a TDMS device with a rectangular cross section, a TDMS device similar to that depicted in FIG. 5 may require the phase closest to the collection plate 4 (in this case, the less dense phase) to have a lower volumetric flow rate to maintain similar flow velocities of the two phases through the chamber.

The following examples demonstrate the basic principles of this invention and various means for employing this invention for magnetic separations.

### Example 1. Effect of Buffer Column Height on Target Cell Purity in a Model System

We have demonstrated that the greater the column height of the non-magnetic phase (i.e., buffer), the greater the purity without a significant change in yield. This effect was demonstrated using ferrofluid-labeled HPB cells (CD3+ cell line) spiked into RBC (15% hematocrit) and placed into microtiter wells with different column heights, over which buffer was layered of reciprocal column heights such that the total column heights of the two-phase systems were the same. As shown in the data below, the greater the column height of the non-magnetic phase (i.e., buffer), the more pure the product.

| % Sample | % Buffer | Product |
|---|---|---|
| Height of Total | Height of Total | Purity |
| 30% | 70% | 97.3% |
| 60% | 40% | 89.5% |
| 90% | 10% | 69.9% |

### Example 2. Effect of Buffer Column Height on Target Cell Purity in Leukapheresis Product

Leukapheresis product was labeled with anti-CD3 and subsequently labeled with ferrofluid. The suspension of magnetically labeled target cells and non-magnetically labeled non-target cells was diluted two-fold and placed in a rectilinear open-top vessel with interior dimensions of 1.0 cm wide×4.0 cm long×1.5 cm tall. In one case, 3 mL of labeled cell suspension was added to the vessel, followed by 3 mL of buffer layered on top (i.e., sample constituted 50% of the column height). In another case, 1.5 mL of labeled cell suspension was added, followed by 4.5 mL of buffer layered on top (i.e., sample constituted 25% of the column height). A cover slip was placed on top of each vessel, above which a magnetic device with a strong upward-pulling magnetic gradient was positioned. After a 10 min separation, the cover slip and the magnetic device were lifted off the vessel in tandem and rotated 180°. The cover slip was then removed from the magnetic device to retrieve the captured cells. By performing flow cytometry on the recovered cells, it was determined that the purity of the product (i.e., the % CD3+) was 97.8% for the 50% column height sample and 99.0% for the 25% column height sample.

### Example 3. Flowing Different Density Liquids Through a TDMS Device without Mixing

Elongated separation chambers, similar in concept to that depicted in FIG. 3, were fabricated by gluing plastic squares to the open end of clear plastic cuvettes with a square cross section (inside dimensions: 1.0×1.0×4.35 cm). For each such chamber, two 3/32″ holes were drilled in each end, positioned as in FIG. 3. Ports, fashioned by cutting small open-ended cones from pipette tips, were epoxied over the holes so that appropriate micro-bore tubing could be attached to each port. To test the flow parameters of two solutions with different densities in such chambers, the chambers were connected via the four ports to a four-channel peristaltic pump (Minipuls 2, Gilson). With the first pump channel, a dense solution (5-10% w/v sucrose) was pumped from a source into the lower inlet port of the chamber until the chamber was filled approximately halfway. At this point, the second pump channel was connected to the lower outlet port to pump the dense solution out of the chamber and into a collection vessel at the same rate it was being pumped in, thus keeping the dense liquid level constant. Next, less dense solution was slowly pumped via the third pump channel from a source into the upper inlet port of the chamber so as to layer onto the lower denser liquid. When the chamber was filled, the upper outlet port of the chamber was connected to the fourth pump channel so that the four pump channels could be run simultaneously whereby the two phases entered the chamber at the same rate from two different sources and were pumped out of the chamber at the same rate to their respective collection vessels.

In order to observe whether mixing of the phases was occurring during their passage through the chamber, sufficient food dye was added to make the liquids distinguishable from one another and make the boundary clearly visible. Using the aforementioned arrangement, it was determined that each solution could be pumped through the chamber at at least 1.5 mL/min with no signs of mixing or boundary disturbance for considerable lengths of time (tested up to 20 min). Since the obtainable flow rates were in excess of that required for a magnetically labeled entity to move from one phase to the extreme side of the other phase, it was apparent that flowing two solutions of different densities through such a chamber was achievable.

### Example 4. Continuous Capture of Magnetic Nanoparticles

The chamber and peristaltic pump arrangement described in Example 3 was used for these experiments. The magnetic nanoparticles employed were proprietary ferrofluids prepared by a modification of Liberti et al. (U.S. Pat. No. 6,120,856). These materials have a mean diameter of 130 nm and are composed of quasi-spherical cores of magnetite (ca. 115 nm) coated with layers of either human or bovine serum albumin. They are highly magnetic, comprising greater than 80% magnetic mass.

Ferrofluid concentrations of 1.0, 2.5, 5.0 and 10 μg/mL were prepared in an isotonic cell buffer with added protein (1% w/v BSA). For experiments wherein the magnetic gradient pulled magnetic entities downwards, the above solutions were layered on top of a similar buffer containing 10% w/v sucrose. Chambers were loaded with layered solutions as described previously. When distinct and unperturbed flowing layers were observed, samples were introduced into the top flowing layer. Initial pumping rates for both solutions were 800 μL/min; hence, the dwell time of a nanoparticle in the chamber was about 5.5 min. At that rate, in all cases, ferrofluid was collected on the bottom of the chamber after traversing approximately 25% of the chamber distance. As expected, by increasing the flow rate, ferrofluid was collected after traversing slightly more than half the chamber distance. At a flow rate of 4 mL/min, the design of the inlet port created turbulence; however, with appropriate design modifications, rates of at least that high are feasible as the barrier between the two phases remained mostly intact.

### Example 5. TDMS Device for Continuous Magnetic Separation of CD34+ Stem Cells

In the invention disclosed herein, there are several parameters that can be controlled to pull magnetically labeled entities from a more dense solution to a less dense solution, or vice versa. Furthermore, those parameters can be tuned such that magnetically labeled entities that are pulled into the “clean” solution (i.e., the phase which is initially devoid of cells) exit the chamber rather than collecting within the chamber. In the case of an entity such as a mammalian cell, those adjustable parameters include dwell time of the magnetically labeled entities in the chamber (determined by solution flow rates and length of the chamber), solution properties (density and viscosity of the solutions), gradient of the magnetic field strength, and magnetic loading of the labeled entities.

For a particular cell type (e.g., a CD34+ human stem cell), this would be accomplished by labeling a cell suspension (obtained by bone marrow aspiration or from mobilized apheresis product) with anti-CD34 by appropriate incubation, washing to remove unbound antibody, and magnetically labeling with an appropriate magnetic nanoparticle (e.g., a ferrofluid coated with rat anti-mouse IgG or, alternatively, a ferrofluid coated with streptavidin if the anti-CD34 is biotinylated). Employing a chamber similar to that described here—4.35 cm in length—and flow rates through the chamber of between 0.2 and 3.0 mL/min, the degree of magnetic labeling that would prevent the target cells from being collected within the chamber would be determined. This might require decreasing or increasing the length of the chamber. Nonetheless, by controlling simple physical parameters, the appropriate conditions will be determined to permit collection of CD34+ cells with the “clean” solution exiting the chamber.

### Example 6. Use of a TDMS Device as an Analytical Tool

The mode of operation of this invention provides the potential to perform in-depth analysis of magnetic materials or materials that are magnetically labeled. Most magnetic separations are binary in nature; that is, in a quadrupole separation, entities either collect on the inner walls of the container or they do not. On the other hand, in TDMS devices, the distance a magnetic entity travels before being captured on the collection surface of the chamber provides information about its magnetic character. For example, if there is a distribution of magnetic labeling due to a distribution of receptor density for a given cell type, that would manifest in the way cells are magnetically collected; that is, a more narrow band on the collection surface would indicate a more uniform distribution of receptors, providing that the magnetic labeling is at saturation.

That would also be applicable for examining the heterogeneity of magnetic nanoparticles. A tight distribution of particle size (i.e., magnetic moment) would be indicated by a narrow band on the collection surface in a device and system so described by this invention. In the case of ferrofluid prepared in our laboratories, our manufacturing process typically yields nanoparticles with a mean size of 130 nm. However, we are aware that small particles (50-90 nm) are also produced in the process, which could be detected using a TDMS device.

Using the device and system described in this invention, two ferrofluid solutions—one having a distribution where 97% of particles are 135 nm and 3% are 80 nm, and a second having a distribution where 65% of particles are 135 nm and 35% range from 50-90 nm—could be tested. The experiments would be carried out using flow rates of 1.5 mL/min with the test ferrofluids in the upper, less dense solution. The distribution of ferrofluid collected on the bottom surface of the chamber would be expected to show a region of narrower deposition for the former sample versus a broader deposition for the more polydisperse sample (i.e., mirroring the size distribution obtained by particle size analysis). Hence, this invention could be used as an analytical tool.

There is considerable utility in this invention. It can be used to capture targets on a surface such that they can be recovered from that surface if that is desired, or they can be maintained on the surface and subjected to various treatments to permit a variety of subsequent analyses. Alternatively, this invention can be used to separate target entities from a complex mixture without the need to capture the target entities by adjusting either flow rate and/or magnetic gradient such that magnetically diverted target entities flow out of the chamber rather than being retained therein. It is noteworthy that samples that might contain rare events, such as circulating tumor cells (CTC), would benefit from this invention either by collecting cells outside the chamber or on a collection surface within the chamber because there is essentially no limit to how much sample can be processed using a TDMS device. This could be critically important in applications that use the presence and/or frequency of CTC as a diagnostic or prognostic indicator, wherein a significant quantity of blood must be processed in order to capture a reasonable number of CTC.

In the systems described in this disclosure, target entities are magnetically labeled, separated, and recovered (i.e., positive selection). It should be understood that non-target entities can be magnetically labeled, separated from the non-magnetically labeled target entities, followed by recovery of the latter population (i.e., negative selection). This might be desirable if recovery of “untouched” target cells is of benefit (e.g., if target cells might be activated upon magnetic labeling).

In the systems described in this disclosure, there are two layers of differing densities wherein, depending on the apparatus, the mixture from which an entity is magnetically separated can be in either layer. It should be understood that more than two layers can be used when practicing this invention. This might be desirable if specific reactions or other processing steps on magnetically labeled entities passing through one or more layers are of benefit. Such possibilities add to the utility of this invention.

While certain embodiments of the present invention have been described and/or exemplified above, various other embodiments will be apparent to those skilled in the art from the foregoing disclosure. The present invention is, therefore, not limited to the particular embodiments described and/or exemplified, but is capable of considerable variation and modification without departure from the scope and spirit of the appended claims.

A number of patent and non-patent publications are cited herein in order to describe the state of the art to which this invention pertains. The entire disclosure of each of these publications is incorporated by reference herein.

Furthermore, the transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step, or material. The term “consisting of” excludes any element, step, or material other than those specified in the claim and, in the latter instance, impurities ordinarily associated with the specified material(s). The term “consisting essentially of” limits the scope of a claim to the specified elements, steps, or material(s) and those that do not materially affect the basic and novel characteristic(s) of the claimed invention. All devices, device components, and methods described herein that embody the present invention can, in alternate embodiments, be more specifically defined by any of the transitional terms “comprising,” “consisting essentially of,” and “consisting of.”

What is claimed is:

1. A method of separating magnetically labeled entities from a mixture that also includes non-magnetically labeled entities, wherein said magnetically labeled entities are recoverable in a highly purified state without re-suspension or washing thereof, said method comprising:
a) providing within a vessel a first layer of predetermined density initially comprising said magnetically labeled entities and said non-magnetically labeled entities;
b) providing within said vessel at least one other layer of a substantially different density from that of said first layer, with each adjacent layer having an interface therebetween;
c) providing a collection substrate in contact with a surface of said layer furthest from said first layer and opposite said interface between said layer furthest from said first layer and said adjacent layer; and
d) applying a magnetic field gradient effective to selectively transport said magnetically labeled entities through each said interface, due to said substantially different density between said first layer and said adjacent layer, thereby separating said magnetically labeled entities in a highly purified state from said non-magnetically labeled entities, without performing another active separation step.

2. The method of claim 1, wherein said collection substrate comprises a capture agent that functions to immobilize said entities upon contact with said collection substrate.

3. The method of claim 1, further comprising recovering said highly purified magnetically labeled entities.

4. The method of claim 1, wherein said entities are selected from the group consisting of cells, viruses, organelles, proteins, protein complexes, peptides, chromatin, nucleic acids, oligonucleotides, carbohydrates, lipids, synthetic polymers and any combination thereof.

5. The method of claim 1, wherein said entities are magnetically labeled with magnetic particles.

6. The method of claim 5, wherein said magnetic particles have an average size of about 10 nm to about 250 nm.

7. The method of claim 5, wherein said magnetic particles have an average size of about 250 nm to about 5 μm.

8. The method of claim 1, wherein said first layer is of a higher density than said adjacent layer, causing said first layer to be positioned below said adjacent layer, and said magnetic field gradient is applied from a magnetic field source superposed over said layers.

9. The method of claim 1, wherein said first layer is of a lower density than said adjacent layer, causing said first layer to be positioned above said adjacent layer, and said magnetic field gradient is applied from a magnetic field source disposed beneath said layers.

10. The method of claim 1, wherein at least one said layer comprises salt and a buffering agent which is effective to control osmolarity and pH.

11. The method of claim 1, wherein the density of at least one said layer is controlled by incorporating therein at least one additive selected from the group consisting of sucrose, iodixanol, iohexol, Ficoll™ PM400, or the like which is effective to control solution density.

12. The method of claim 1, wherein at least one said layer comprises at least one additive selected from the group consisting of a fluorescent-staining agent, a cell-lysing agent, or a fixative agent.

13. The method of claim 1, wherein said layers are substantially static.

14. The method of claim 1, wherein at least one said layer is convectively transported through the vessel in a direction that is approximately orthogonal to the applied magnetic field gradient.

15. A system for separating magnetically labeled entities from a mixture that also includes non-magnetically labeled entities, wherein said magnetically labeled entities are recoverable in a highly purified state without re-suspension or washing thereof, said system comprising, in combination:
a) a vessel that contains:
i) a first layer of predetermined density initially comprising said magnetically labeled entities and said non-magnetically labeled entities; and
ii) at least one other layer of a substantially different density from that of said first layer, with each adjacent layer having an interface therebetween;
b) a collection substrate in contact with a surface of said layer furthest from said first layer and opposite said interface between said layer furthest from said first layer and said adjacent layer; and
c) a magnetic field source effective to apply a magnetic field gradient within said vessel and selectively transport said magnetically labeled entities through each said interface, due to said substantially different density between said first layer and said adjacent layer, thereby separating said magnetically labeled entities in a highly purified state from said non-magnetically labeled entities, without performing another active separation step.

16. The system of claim 15, wherein said collection substrate comprises a capture agent that functions to immobilize said entities upon contact with said collection substrate.

17. The system of claim 15, wherein said entities are selected from the group consisting of cells, viruses, organelles, proteins, protein complexes, peptides, chromatin, nucleic acids, oligonucleotides, carbohydrates, lipids, synthetic polymers and any combination thereof.

18. The system of claim 15, wherein said entities are magnetically labeled with magnetic particles.

19. The system of claim 18, wherein said magnetic particles have an average size of about 10 nm to about 250 nm.

20. The system of claim 18, wherein said magnetic particles have an average size of about 250 nm to about 5 μm.

21. The system of claim 15, wherein said first layer is of a higher density than said adjacent layer, such that said first layer is positioned below said adjacent layer, and said magnetic field gradient is applied from said magnetic field source superposed over said layers.

22. The system of claim 15, wherein said first layer is of a lower density than said adjacent layer, such that said first layer is positioned above said adjacent layer, and said magnetic field gradient is applied from said magnetic field source disposed beneath said layers.

23. The system of claim 15, wherein at least one said layer comprises salt and a buffering agent which is effective to control osmolarity and pH.

24. The system of claim 15, wherein the density of at least one said layer is controlled by incorporating therein at least one additive selected from the group consisting of sucrose, iodixanol, iohexol, Ficoll™ PM400, or the like which is effective to control solution density.

25. The system of claim 15, wherein at least one said layer comprises at least one additive selected from the group consisting of a fluorescent-staining agent, a cell-lysing agent, or a fixative agent.

26. The system of claim 15, wherein said layers are substantially static.

27. The system of claim 15, wherein at least one said layer is convectively transported through the vessel in a direction that is approximately orthogonal to the applied magnetic field gradient.
